(19) United States
(12) Patent Application Publication
(10) Pub. No.: US 2009/0208410 A1
(43) Pub. Date: Aug. 20, 2009

(54) ED-B FIBRONECTIN AS STRATIFICATION MARKER FOR ANTI-TUMOR DRUGS

(76) Inventors: Dietmar BERNDORFF, Berlin, DE (US); Ludger Dinkelborg, Berlin, DE (US); Tobias Heinrich, Berlin, DE (US)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/211,198

(22) Filed: Sep. 16, 2008

Related U.S. Application Data
(60) Provisional application No. 60/972,868, filed on Sep. 17, 2007.

(30) Foreign Application Priority Data
Sep. 17, 2007 (EP) 07018213.4

(51) Int. Cl. A61K 51/10; C12N 5/08; A61K 39/395; C07D 207/40; A61P 35/00; A61K 103/10; A61K 101/02

(52) U.S. Cl. 424/1.49; 435/377; 424/139.1; 548/546

(57) ABSTRACT

The present invention relates to a method for stratifying tumor cells comprising the steps: (a) providing a sample comprising tumor cells to be analysed, (b) contacting said sample with an ED-B fibronectin-specific molecule and assessing the binding thereof to tumor cells in said sample, and (c) stratifying said tumor cells based on the assessment of step (b).

This application claims the benefit of the filing date of U.S. Provisional Application Ser. No. 60/972,868 filed Sep. 17, 2007.

### DESCRIPTION

The present invention refers to the use of molecules for the extra-domain B (ED-B) of fibronectin, for example of labeled antibodies or antibody fragments against the ED-B domain as diagnostic reagents for stratifying a tumor with regard to its sensitivity to anti-tumor drugs and particularly as stratification marker for EGF/EGFR antagonists.

Inhibitors of the epidermal growth factor receptor (EGFR) are used in tumor therapy, particularly for the treatment of patients with non-small-cell lung carcinoma (NSCLC). The sensitivity of EGFR-containing human NSCLC cell lines to EGFR inhibition is dependent on the degree to which they have undergone an epithelial to mesenchymal transition (EMT). NSCLC cell lines which express the epithelial cell junction protein E-cadherin show a greater sensitivity to EGFR inhibition compared to cell lines having undergone EMT (Thomson et al., Cancer Res. 65 (2005), 9455-9462).

Further, it was found that the clinical activity of EGFR antagonists in NSCLC patients may be predicted by determination of EMT in human tumor cells (Yauch et al., Clin. Cancer Res. 11 (2005), 8686-8698).

EMT is characterized by loss of epithelial markers, e.g. E-cadherin, and gain of mesenchymal markers, e.g. vimentin. EMT leads to de-differentiation, loss of adhesive constraint and enhanced mutility and invasion. These properties are hallmarks of increased malignancy. Thus, EMT provides a mechanism for carcinoma cells to acquire this more aggressive phenotype (Lee et al., J. Cell. Biol. 172 (2006), 973-981; Christiansen and Rajasekaran, Cancer Res. 66 (2006), 8319-8326). Detection of EMT by routine analytic methods is, however, difficult, because this process probably occurs transiently in discrete areas within tumors (Gotzman et al., Mutation Res. 566 (2004)₉-20).

Thus, it was an object of the present invention to provide a reliable and sensitive method which allows determination of epithelial versus mesenchymal phenotype in human tumor cells or tissues or other cells or tissues of interest.

In a first aspect the present invention refers to a method for stratifying a tumor cell or tumor tissue comprising the steps of

(a) contacting an ED-B fibronectin-specific molecule with a tumor cell or a tumor tissue and assessing the binding of the ED-B fibronectin-specific molecule to the tumor cell or the tumor tissue, and
(b) stratifying said tumor cell or tumor tissue based on the assessment of step (a).

In a further aspect the present invention refers to a method of detecting, particularly of imaging, epithelial mesenchymal transition (EMT) in a cell or tissue, particularly in a tumor cell or tumor tissue, comprising the steps of

(a) contacting an ED-B fibronectin-specific molecule with a cell or a tissue and assessing the binding of the ED-B fibronectin-specific molecule to the cell or tissue, and
(b) identifying said cell or tissue as epithelial-type cell or tissue or as mesenchymal-type cell or tissue based on the assessment of step (a).

In a further aspect, the present invention relates to a method for stratifying a tumor cell or tumor tissue as epithelial-type tumor cell or tissue if substantial binding of a ED-B fibronectin-specific molecule is observed or as mesenchymal-type tumor cell or tissue if no substantial binding of a ED-B fibronectin-specific molecule is observed comprising the steps of

(a) contacting an ED-B fibronectin-specific molecule with a tumor cell or a tumor tissue and assessing the binding of the ED-B fibronectin-specific molecule to the tumor cell or the tumor tissue, and
(b) stratifying said tumor cell or tumor tissue based on the assessment of step (a).

In a particularly preferred embodiment, substantial binding of said ED-B fibronectin-specific molecule indicates sensitivity to anti-tumor treatment comprising administration of an EGF/EGFR antagonist.

Still a further aspect of the present invention refers to the use of an ED-B fibronectin-specific molecule for the manufacture of an agent for stratifying a tumor cell or tumor tissue as being sensitive or refractory against treatment.

In a further embodiment, the invention relates to the use of an ED-B fibronectin-specific molecule for a method of stratifying a tumor cell or a tumor tissue as being sensitive or refractory against treatment an EGF/EGFR antagonist.

Still a further aspect of the present invention refers to the use of an ED-B fibronectin-specific molecule for the manufacture of an agent for stratifying a tumor cell or tumor tissue as being sensitive or refractory against treatment with an EGF/EGFR antagonist. In a preferred embodiment, substantial binding of an ED-B fibronectin-specific molecule to the tumor cell or tumor tissue is indicative for a patient being as being sensitive to treatment with an EGF/EGFR antagonist.

Still a further aspect of the present invention refers to the use of an ED-B fibronectin binding molecule for the manufacture of an agent for detecting epithelial mesenchymal transition. Still a further aspect of the present invention refers to the use of an ED-B fibronectin binding molecule in a method of detecting epithelial mesenchymal transition. In a preferred embodiment, substantial binding of an ED-B fibronectin-specific molecule to the tumor cell or tumor tissue is indicative for tissue which has not or not yet undergone the epithelial mesenchymal transition.

Still a further aspect of the present invention refers to a composition or kit for stratifying a tumor cell or tumor tissue comprising an ED-B fibronectin-specific binding molecule conjugated with a detectable labelling group.

Still a further aspect of the present invention refers to a composition or kit for detecting epithelial mesenchymal transition comprising an ED-B fibronectin-specific binding molecule conjugated with a detectable labelling group.

Still a further aspect of the present invention relates to a composition or kit for treating a tumor comprising an ED-B fibronectin-specific binding molecule conjugated with a detectable labelling group and an EGF/EGFR antagonist.

In a preferred embodiment, the inventions relates to the use of a composition or kit comprising an ED-B fibronectin-specific binding molecule conjugated with a detectable labelling group and an EGF/EGFR antagonist, in a method of treating tumors.

In a particularly preferred embodiment, the ED-B fibronectin-specific binding molecule conjugated with a detectable labelling group is AP39 conjugated with a detectable labelling group, in particular ^{99m}Tc-labelled AP39.

Still a further aspect of the present invention refers to a composition or kit for treating a tumor patient comprising stratifying a tumor cell or tumor tissue followed by treatment of a patient identified as being sensitive to treatment with an EGF/EGFR antagonist, wherein the kit comprises an ED-B fibronectin-specific binding molecule conjugated with a detectable labelling group and an EGF/EGFR antagonist.

Still a further aspect of the present invention refers to a composition or kit for treating a tumor patient comprising detecting epithelial mesenchymal transition followed by treatment of a patient which has not undergone epithelial mesenchymal transition, wherein the kit comprises an ED-B fibronectin-specific binding molecule conjugated with a detectable labelling group and an EGF/EGFR antagonist.

In a further embodiment, the present invention relates to a method of treating a tumour patient, comprising the steps of
(a) administering to a tumor patient a diagnostically effective amount of an ED-B fibronectin-specific binding molecule conjugated with a detectable labelling group
(b) determining whether the patient is sensitive to treatment with an EGF/EGFR antagonist by determining whether an ED-B fibronectin-specific binding molecule binds substantially to tissue, and
(c) administering to the tumor patient a therapeutically effective amount of an EGF/EGFR antagonist if the patient shows substantial binding of the ED-B fibronectin-specific binding molecule.

According to the present invention it was surprisingly found that determination of ED-B fibronectin can be used for the selection or stratification of a tumor cell or tumor tissue and/or for the detection of EMT. Binding of tumor cells or tumor tissue to ED-B specific molecules indicates sensitivity for anti-tumor drugs, such as EGF/EGFR antagonists and/or epithelial phenotype. Lack of binding or reduced binding of tumor cells or tumor tissue to ED-B specific molecules indicates resistance against anti-tumor drugs, such as EGF/EGFR antagonists and/or mesenchymal phenotype. Thus, the present invention is particularly suitable for the selection or stratification of tumor patients and their sensitivity towards anti-tumor treatment, such as treatment with EGF/EGFR antagonists.

Molecules specific for the ED-B domains of fibronectin, a sequence of 91 amino acids, which is inserted by alternative splicing into the fibronectin molecule (Castellani et al. (1994), Int. J. Cancer 59, 612-18), are already described in WO 97/45544, WO 01/62800, WO 03/055917, WO 03/07649 and WO 2005/0223318, which are herein incorporated by reference. Preferred binding molecules are molecules that bind directly and specifically to the ED-B domains, such as, for example, antibodies against the ED-B domains or fragments of such antibodies, for example antibody fragments that can be obtained by proteolytic cleavage, e.g. Fab-, Fab′- F(ab₂) fragments etc., or recombinant antibody fragments, e.g. single-chain Fv-fragments. The ED-B-binding molecules are preferably used as conjugates with labeling groups that are suitable for diagnostic applications.

A preferred embodiment of the invention relates to the use of antibody L19 or fragments of this antibody (L19 derivatives), which are present as conjugates with labeling groups.

L19 is the scFv fragment (scFv: single chain variable antibody fragment) of a monoclonal antibody against the extra-domain B (ED-B) of fibronectin and has the following amino acid sequence (SEQ ID NO. 1):

| | | |
|---|---|---|
| (VH): | | |
| EVQLLESGGG | LVQPGGSLRL | SCAASGFTFS |
| SFSMSWVRQA | PGKGLEWVSS | ISGSSGTTYY |
| ADSVKGRFTI | SRDNSKNTLY | LQMNSLRAED |
| TAVYYCAKPF | PYFDYWGQGT | LVTVSS |
| (Linker): | | |
| GDGSSGGSGG | ASTG | |
| (VL): | | |
| EIVLTQSPGT | LSLSPGERAT | LSCRASQSVS |
| SSFLAWYQQK | PGQAPRLLIY | YASSRATGIP |
| DRFSGSGSGT | DFTLTISRLE | PEDFAVYYCQ |
| QTGRIPPTFG | QGTKVEIK | |

Especially preferred are L19 derivatives comprising
(aa) at least one antigen binding site for the extra-domain B (ED-B) of fibronectin comprising the complementarity-determining regions HCDR3 and/or LCDR3, shown in Table 1, or a variant thereof, which exhibits a deletion, insertion and/or substitution of up to 5 amino acids in the HCDR3 region and up to 6 amino acids in the LCDR3 region, whereby the antigen binding site exhibits the same function as the native L19 shown in SEQ ID NO. 1,
(ab) at least one antigen binding site for the extra-domain B (ED-B) of fibronectin comprising the complementarity-determining regions HCDR1, HCDR2, HCDR3, LCDR1, LCDR2 and LCDR3, shown in Table 1, or a variant thereof, which exhibits a deletion, insertion and/or substitution of up to 3 amino acids in the HCDR1 region, of up to 8 amino acids in the HCDR2 region, of up to 5 amino acids in the HCDR3 region, of up to 6 amino acids in the LCDR1 region, of up to 4 amino acids in the LCDR2 region and of up to 6 amino acids in the LCDR3 region, whereby the antigen binding site exhibits the same function as the native L19 shown in SEQ ID NO. 1, or
(ac) at least one antigen binding site for the extra-domain B (ED-B) of fibronectin comprising the sequence of the native L19, shown in SEQ ID NO. 1, or a variation thereof, which exhibits a deletion, insertion and/or substitution of up to 30 amino acids, whereby the antigen binding site exhibits the same function as the native L19 shown in SEQ ID NO. 1, and optionally
(ba) an amino acid sequence Xaa₁-Xaa₂-Xaa₃-Cys (SEQ ID NO. 2),
   whereby Xaa₁, Xaa₂, and Xaa₃, independently of one another, represent any naturally occurring amino acid,
(bb) an amino acid sequence Xaa₁-Xaa₂-Xaa₃-Cys-Xaa₄ (SEQ ID NO. 3), whereby Xaa₁, Xaa₂, Xaa₃, and Xaa₄, independently of one another, represent any naturally occurring amino acid,
(bc) an amino acid sequence (His)ₙ (SEQ ID NO. 4), whereby n is an integer from 4 to 6, or
(bd) an amino acid sequence that comprises the sequence shown in SEQ ID NO. 5, SEQ ID NO. 6 or SEQ ID NO. 7,
   whereby the C-terminus of (aa), (ab), or (ac) is optionally bonded via a peptide bond to the N-terminus of (ba), (bb), (bc) or (bd).

Within the scope of this invention, the labeled L19 derivative comprises an N-terminal antigen binding site for the extra-domain B (ED-B) of fibronectin selected from the antigen binding sites (aa), (ab) or (ac) and optionally a C-terminal amino acid sequence selected from the amino acid sequences (ba), (bb), (bc) or (bd), whereby the antigen binding site exhibits the same function as the native L19 shown in SEQ ID NO. 1.

According to this invention, the antigen binding sites for the extra-domain B (ED-B) of fibronectin of the labeled L19 derivative (aa) or (ab) comprise the complementarity-determining regions HCDR3 and/or LCDR3 or HCDR1, HCDR2, HCDR3, LCDR1, LCDR2 and LCDR3, shown in Table 1. Within the scope of this invention, the complementarity-determining regions HCDR1, HCDR2, HCDR3, LCDR1, LCDR2 and LCDR3 are defined as follows:

TABLE 1

| | CDR Length⁽²⁾ | | Maximum |
|---|---|---|---|
| | (in Amino | | (Preferred) |
| Region⁽¹⁾ | Acids) | Sequence | Variations |
| HCDR1 | 5 | S F S M S | 3 (2, 1) |
| | | (SEQ ID NO. 8) | |
| HCDR2 | 17 | S I S G S S G T T Y | 8 (7, 6, 5, 4, 3, 2, 1) |
| | | Y A D S V K G | |
| | | (SEQ ID NO. 9) | |
| HCDR3 | 7 | P F P Y F D Y (SEQ | 5 (4, 3, 2, 1) |
| | | ID NO. 10) | |
| LCDR1 | 12 | R A S Q S V S S S | 6 (5, 4, 3, 2, 1) |
| | | F L A (SEQ ID NO. | |
| | | 11) | |
| LCDR2 | 7 | Y A S S R A (SEQ | 4 (3, 2, 1) |
| | | ID NO. 12) | |
| LCDR3 | 10 | C Q Q T G R I P P T | 6 (5, 4, 3, 2, 1) |
| | | (SEQ ID NO. 13) | |

⁽¹⁾HCDRx: Complementarity-determining region × the heavy antibody chain; LCDRx: complementarity-determining region × the light antibody chain.
⁽²⁾CDR length: Length of the complementarity-determining region.

In addition to the complementarity-determining regions defined in Table 1, the antigen binding sites for the extra-domain B (ED-B) of fibronectin of the labeled L19 derivative (aa) or (ab) can also comprise variants of these regions. According to the invention, a variant of the HCDR1 region comprises a deletion, insertion and/or substitution of up to 3 amino acids in the HCDR1 region, i.e., a deletion, insertion and/or substitution of 1, 2 or 3 amino acids relative to the sequence (SEQ ID NO. 8) shown in Table 1. A variant of the HCDR2 region comprises a deletion, insertion and/or substitution of up to 8 amino acids in the HCDR2 region, i.e., a deletion, insertion and/or substitution of 1, 2, 3, 4, 5, 6, 7 or 8 amino acids relative to the sequence (SEQ ID NO. 9) shown in Table 1. Moreover, a variant of the HDCR3 region comprises a deletion, insertion and/or substitution of up to 5 amino acids in the HCDR3 region, i.e., a deletion, insertion and/or substitution of 1, 2, 3, 4 or 5 amino acids relative to the sequence (SEQ ID NO. 10) shown in Table 1. A variant of the LCDR1 region, however, comprises a deletion, insertion and/or substitution of up to 6 amino acids in the LCDR1 region, i.e., a deletion, insertion and/or substitution of 1, 2, 3, 4, 5 or 6 amino acids relative to the sequence (SEQ ID NO. 11) shown in Table 1. In addition, a variant of the LCDR2 region comprises a deletion, insertion and/or substitution of up to 4 amino acids in the LCDR2 region, i.e., a deletion, insertion and/or substitution of 1, 2, 3 or 4 amino acids relative to the sequence (SEQ ID NO. 12) shown in Table 1. A variant of the LCDR3 region comprises a deletion, insertion and/or substitution of up to 6 amino acids in the LCDR3 region, i.e., a deletion, insertion and/or substitution of 1, 2, 3, 4, 5 or 6 amino acids relative to the sequence (SEQ ID NO. 13) shown in Table 1.

According to this invention, the antigen binding site for the extra-domain B (ED-B) of fibronectin of the labeled L19 derivative (ac) comprises the sequence of native L19, shown in SEQ ID NO. 1, or a variation thereof, which exhibits a deletion, insertion and/or substitution of up to 30 amino acids, i.e., a deletion, insertion and/or substitution of 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids relative to the sequence shown in SEQ ID NO. 1.

The amino acid sequences (ba), (bb) or (bc) of the labeled L19 derivative comprise the sequences Xaa₁-Xaa₂-Xaa₃-Cys (SEQ ID NO. 2), Xaa₁-Xaa₂-Xaa₃-Cys-Xaa₄ (SEQ ID NO. 3) or (HIS)ₙ (SEQ ID NO. 4).

In a preferred embodiment of this invention, the amino acid sequence (ba) Xaa₁-Xaa₂-Xaa₃-Cys (SEQ ID NO. 2) is the sequence Gly-Gly-Gly-Cys (SEQ ID NO. 14) or Gly-Cys-Gly-Cys (SEQ ID NO. 15). Especially preferred is the sequence Gly-Gly-Gly-Cys (SEQ ID NO. 14).

In another preferred embodiment of this invention, the amino acid sequence (bb) Xaa₁-Xaa₂-Xaa₃-Cys-Xaa₄ (SEQ ID NO. 3) is the sequence Gly-Gly-Gly-Cys-Ala (SEQ ID NO. 16) or Gly-Cys-Gly-Cys-Ala (SEQ ID NO. 17). Especially preferred is the sequence Gly-Gly-Gly-Cys-Ala (SEQ ID NO. 16).

In another preferred embodiment of this invention, the amino acid sequence (bc) (His)ₙ (SEQ ID NO. 4) is the sequence (His)₆ with n equal to 6 (SEQ ID NO. 18).

In another preferred embodiment of this invention, the N-terminus of (aa), (ab) or (ac) is optionally connected via a peptide bond to the C-terminus of a linker amino acid sequence. The linker amino acid sequence preferably has a length of up to 30 amino acids, preferably up to 25 amino acids, and especially preferably up to 22 amino acids. Especially preferred is the linker amino acid sequence, which is the sequence shown in SEQ ID NO. 19.

According to this invention, especially preferred labeled L19 derivatives comprise the sequences shown in SEQ ID NO. 1 (native L19), SEQ ID NO. (AP38), SEQ ID NO. 21 (AP39), SEQ ID NO. 22 (L19-GlyCysGlyCys), SEQ ID NO. 23 (L19-GlyCysGlyCysAla), SEQ ID NO. 24 (ZK225293), SEQ ID NO. 25 (ZK217691/217695), SEQ ID NO. 26 (ZK210917) and SEQ ID NO. 27 (ZK248219/248220). Especially preferred is AP39.

^{99m}Tc-labelled AP39 is preferably applied to the patient by parenteral or intravenous administration, more preferably by intravenous injection. The human dose is typically in the range of about 0.01 to 10 mg, preferably in the range of about 0.1. to 1 mg per patient.

The binding molecule for the ED-B domain is preferably present in the form of a conjugate with a labelling group. As labelling groups, all labelling substances that are suitable for diagnostic applications, especially diagnostic applications in vivo, may be used, for example radiolabelling substances for radioactive detection, e.g. radioactive imaging methods, or for non-radioactive detecting, e.g. non-radioactive imaging methods, such as labelling substances that are suitable for optical imaging or magnetic resonance imaging. In an especially preferred embodiment, the labelling group is a labelling group suitable for SPECT (Single Photon Emission Computed Tomography), in particular the radioisotope ^{99m}Tc. In a further especially preferred embodiment, the labelling group is a labelling group suitable for PET (Positron Emission Tomography), in particular the radioisotope ¹⁸F. The application of the methods and uses of the present invention in PET or SPECT is preferred.

The diagnostic application in vivo is particularly preferred, as the present uses and methods also allow diagnosis without taking biopsies, as biopsies are often difficult to take and require surgical intervention. Also, ED-B fibronectin is a stromal extra-cellular matrix protein and accessible for in vivo imaging procedures. It is located abluminally and extracellularly at the neovasculature of tumors.

Processes for introducing labeling substances in polypeptides, peptides and especially scFv fragments are well known in the prior art. The binding molecule is preferably labeled with a radioisotope, e.g., a radioisotope of iodine (I), indium (In), Yttrium (Y), technetium (Tc), rhenium (Re), gallium (Ga), bromine (Br) or fluorine (F). Especially preferred are the radioisotopes ¹²⁵I, ¹²³I, ¹²⁴I, ¹³¹I, ¹¹¹In, ¹⁸⁶Re, ¹⁸⁸Re, ⁸⁶Y, ^{94m}Tc, ^{99m}Tc, ⁶⁷Ga, ⁶⁸Ga, ⁷⁶Br or ¹⁸F. Preferred radioisotopes are ^{99m}Tc, ¹⁸F and ¹²³I.

The synthesis of the ¹⁸F-labelled AP39 molecules is described in examples 4 and 5. These ¹⁸F-labelled molecules are in particular suitable for use in PET according to the present invention. AP39 is a derivative of the L19 antibody as described in WO 03/055917.

Thus, in another preferred embodiment, ¹⁸F-labelled AP39, especially selected from [¹⁸F]FBAM-AP39 and [¹⁸F]SFB-AP39, is in particular useful for the methods and uses of the present invention, in particular with PET.

In a preferred embodiment of this invention, an antibody fragment, e.g., an L19 derivative in reduced form, is used. Within the scope of this invention, the term “reduced form” means that the fragment is present in monomeric form and not, for example, in dimeric or multimeric form that is mediated by intermolecular disulfide bridges. The reduced form of the antibody fragment is preferably obtained by adding a suitable reducing agent. Suitable reducing agents are well known in the prior art and comprise TCEP (tris(2-carboxyethyl)phosphine) and 1,4-dimercapto-2,3-butanediols.

In addition, this invention provides that the pharmaceutical composition or kit, in addition to the binding molecule, optionally contains physiologically compatible adjuvants, vehicles and/or diluents. Suitable adjuvants, vehicles and/or diluents are best known to one skilled in the art in the field of pharmaceutical chemistry.

In a preferred embodiment of the present invention tumors are stratified as epithelial-type tumors if substantial binding of the ED-B fibronectin-specific molecule is observed or as mesenchymal-type tumors, if no substantial binding of the ED-B fibronectin-specific molecule is observed. In this context the term “substantial binding” comprises an immunological reaction with a target antigen which can be distinguished over unspecific binding to control cells or control tissue or binding to mesenchymal-type cells. In this context it should be noted that the term “substantial binding” also includes cellular uptake. Substantial binding may be determined directly and qualitatively, by visual inspection or via quantitative or semiquantitative programs for analysis of histological stainings, of SPECT cameras and PET analysis.

The cells or tissues to be analyzed according to the present invention are preferably cells which are capable of undergoing EMT, or tissues comprising cells which are capable of undergoing EMT. More preferably, the cells or tissues are tumor cells or tumor tissues, e.g. lung-cancer cells or tissues, such as NSCLC cells or tissues or cells or tissues from prostate, breast, pancreas, colon, head-and-neck, ovarian, renal, gastric or cervix cancer and other tumor cells or tissues, e.g. as described by Christiansen and Rajasekaran (supra).

Preferably, the cells or tissues are from a human tumor patient. It should be noted that the method of the present invention may be carried out in vitro or in vivo. An in vitro diagnostic method comprises the extracorporeal testing of a cell or tissue sample, e.g. a tumor cell or tumor tissue sample, which is preferably derived from a human patient. The in vivo method comprises administering the ED-B fibronectin specific molecule to a subject, e.g. a human patient or an experimental animal and carrying out a determination, preferably an imaging procedure, in the subject.

The in vivo method of the present invention is preferably carried out by injecting the pharmaceutical composition, which comprises the ED-B-binding molecule, into a vein and/or artery of a patient to be examined and detecting the labeled ED-B-binding molecule that is bound to cells or tissue, e.g. tumor cells or tumor tissue, if present. If a radioisotope-labeled binding molecule is used, the detection can be carried out by scintigraphy, SPECT or PET.

The method of the present invention is particularly suitable for the stratification of patients, preferably tumor patients with regard to the sensitivity toward treatment, preferably toward anti-tumor treatment. The method may be carried out before the start of an anti-tumor treatment of the patient, in order to determine sensitivity toward certain treatment protocols. Alternatively or additionally, the method may also be carried out during anti-tumor treatment of the patient. The anti-tumor treatment preferably comprises administration of an EGF/EGFR antagonist, optionally in combination with the administration of further anti-tumor agents and/or radiation therapy. The EGF/EGFR antagonist may be selected from small molecule EGFR inhibitors, e.g. EGFR kinase inhibitors such as erlotinib or gefitinib. Alternatively, the EGF/EGFR antagonist may be selected from anti-EGFR antibodies, such as cetuximab.

The recommended human dose of cetuximab, as monotherapy or in combination with irinotecan, is 400 mg/m² as an initial loading dose which is intravenously administered. The recommended weekly maintenance dose is 250 mg/m² which is intravenously administered as well. Such dose regimen may typically be applied according to the present invention to a patient, however, also lower or higher doses are possible.

In an especially preferred embodiment the method of the present invention is carried out prior to the start of anti-tumor treatment using an EGFR antagonist, such as erlotinib. Thereby therapy-resistant patients may be distinguished from therapy-sensitive patients, thereby reducing ineffective tumor therapies to a high degree.

Further, the present invention shall be explained in more detail by the following examples.

### EXAMPLE 1

### Production of L19 Derivatives

The production of L19 derivative AP39 is carried out as described in WO 03/055917, to whose content reference is made herein.

### EXAMPLE 2

### Labeling of L19 Derivatives with the Aid of Radioisotopes

The production of the labeled L19 derivative AP39-Tc99m is carried out as described in WO 03/055917, to whose content reference is made herein.

### EXAMPLE 3

### Study of the Binding of Labeled L19 Derivatives to NSCLC Cell Lines

Binding studies were performed in two human NSCLC tumor models in mice: NCI-H292 which is an erlotinib-sensitive epithelial-type model, and Calu-6 which is an erlotinib-resistant mesenchymal-type model.

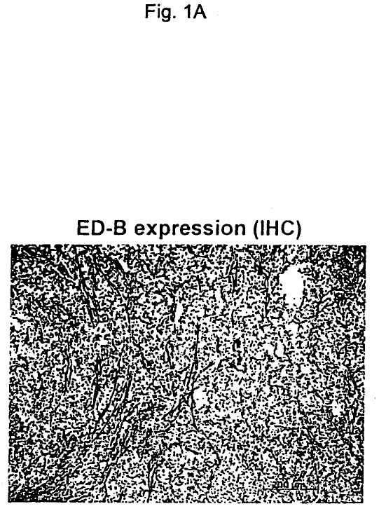

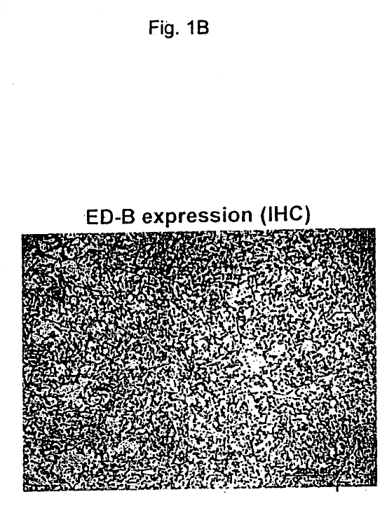

TCEP-reduced AP39 was biotinylated using 3-(N-maleimidopropionyl) biocytin (Sigma). The in vitro binding of biotinylated AP39 to frozen sections (5-10 μm) of NCI-H292 and Calu-6 tumor tissue was determined by utilizing ExtrAvidin (Sigma) and Liquid DAB Substrate Pack (BioGenex) according to the protocols of the manufacturers. A more extensive binding of biotinylated AP39 was observed in NCI-H292 (FIG. 1A) compared to Calu-6 (FIG. 1B) tumor tissue.

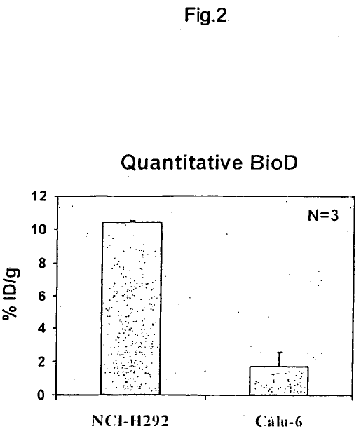

Tc-99m radiolabelled AP39 was injected intravenously in a dose of about 111 kBq into NCI-H292 and Calu-6 tumor bearing nude mice (bodyweight about 30 g). The radioactivity concentration in the tumors was measured ex vivo using a gamma-counter at 1 hour after administration of the substance. It can be gathered that the concentration of Tc-99m-AP39 is substantially higher in NCI-H292 tumor tissue (FIG. 2).

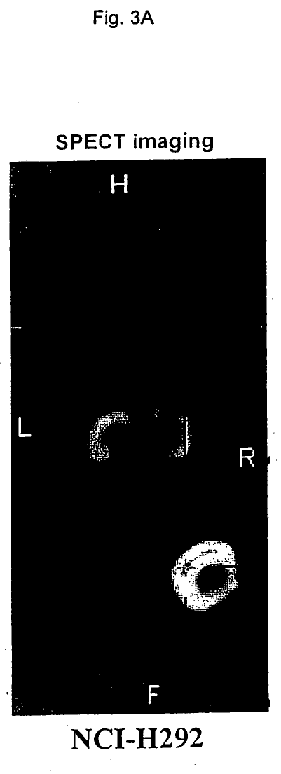

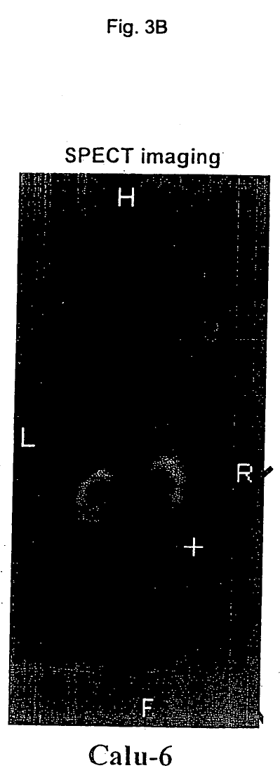

Tc-99m radiolabelled AP39 was injected intravenously in a dose of about 150 MBq into NCI-H292 and Calu-6 tumor bearing nude mice (bodyweight about 30 g). SPECT imaging was carried out at 4 hours after administration of the substance. The result of this investigation shows that the NCI-H292 (FIG. 3A) tumor can be much better depicted by SPECT imaging than the Calu-6 (FIG. 3B) tumor.

### EXAMPLE 4

### Synthesis of N-[6-(4-[¹⁸F]fluorobenzylidene) aminooxyhexyl]maleimide AP39 ([¹⁸F]FBAM-AP39)

[¹⁸F]FBAM was synthesized according to literature (Berndt et al. Nucl Med. Biol. 2007, 34(1), 5-15) in a one-pot synthesis on a GE tracerlab and purified by isocratic semi preparative HPLC (t_{R}=15 min; 65/35 Water/MeCN+0.1% TFA; ACE 5 C18-HL 250*10 mm; 5 μm; Advanced Chromatography Technologies; Cat. No.: ACE 321-2510). In a typical experiment [¹⁸F]FBAM was isolated in amounts between 500 to 1.000 MBq after 75 min in 30-40% radiochemical yield corrected for decay. After HPLC the total volume was reduced by C-18 SPE and [¹⁸F]FBAM was formulated in 2 mL MeCN and dried in a stream of nitrogen at 55° C. until dryness. [¹⁸F]FBAM was dissolved in DMSO/0.05M Tris/NaCl Buffer (pH=7.4) (1/9 (v/v)) and AP39 (100 μg in 90 μL PBS, pH 7.4) was added and incubated for 30 min at 37° C. The conjugation rate was verified by analytical HPLC (t_{R}=13.2 min; TSKgel superSW2000; 300*4.6 mm; 4μ; Toso Biosep; TSK buffer (0.1M Na₂HPO₄/0.1M Na₂SO₄/0.05% NaN₃; pH6.7, isocratic 0.3 ml/min). F-18 labeled AP39 was purified by gel filtration using a Sephadex® NAP 5 cartridge and analyzed by HPLC and electrophoresis. Assessment of immunoreactivity was performed by affinity chromatography according to Birchler et al. (J. Immunol. Methods 1999, 231, 239-248) using a column containing ED-B fibronectin-conjugated Sepharose. Non corrected conjugation yields were between 10 and 15% regarding [¹⁸F]FBAM.

¹⁸F-labelled AP39, especially [¹⁸F]FBAM-AP39, is in particular useful for the methods and uses of the present invention, in particular with PET.

### EXAMPLE 5

### Synthesis of N-succinimidyl 4-[¹⁸F]fluorobenzoate AP39 ([¹⁸F]SFB-AP39)

[¹⁸F]SFB was synthesized according to literature (Kabalka et al., Journal of Labeled Compounds and Radiopharmaceuticals, 2008, 51, 68-71) in a one-pot synthesis on a GE tracerlab and purified by isocratic semi preparative HPLC (t_{R}=16.5 min; 65/35 Water/MeCN+0.1% TFA; ACE 5 C18-HL 250*10 mm; 5 μm; Advanced Chromatography Technologies; Cat. No.: ACE 321-2510). In a typical experiment [¹⁸F]SFB was isolated in amounts between 400 to 800 MBq after 65 min in 30-35% radiochemical yield corrected for decay. After HPLC the volume of [¹⁸F]SFB was reduced by C-18 SPE and [¹⁸F]SFB was formulated in 2 mL MeCN and dried in a stream of nitrogen at 55° C. until dryness. [¹⁸F]SFB was re-dissolved in DMSO/0.1M Borate Buffer (pH=8.6) (1/9 (v/v)) and AP39 (100 μg in 90 μL PBS, pH 7.4) was added. Incubation was performed for 30 min at 37° C. The conjugation rate was verified by analytical HPLC (t_{R}=13.2 min; TSKgel superSW2000; 300*4.6 mm; 4μ; Toso Biosep; TSK buffer (0.1M Na₂HPO₄/0.1M Na₂SO₄/0.05% NaN₃; pH6.7, isocratic 0.3 ml/min). F-18 labeled AP39 was purified by gel filtration using a Sephadex® NAP 5 cartridge and analyzed by HPLC and electrophoresis. Assessment of immunoreactivity was performed by affinity chromatography according to Birchler et al. using a column containing ED-B fibronectin-conjugated Sepharose. Non corrected conjugation yields were between 10 and 12% regarding [¹⁸F]FBAM.

¹⁸F-labelled AP39, especially [¹⁸F]SFB-AP39, is in particular useful for the methods and uses of the present invention, in particular with PET.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding European application No. 07018213.4, filed Sep. 17, 2007, and U.S. Provisional Application Ser. No. 60/972,868, filed Sep. 17, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. A method for stratifying a tumor cell or tumor tissue as epithelial-type tumor cell or tissue if substantial binding of a ED-B fibronectin-specific molecule is observed or as mesenchymal-type tumor cell or tissue if no substantial binding of a ED-B fibronectin-specific molecule is observed comprising the steps of
(a) contacting an ED-B fibronectin-specific molecule with a tumor cell or a tumor tissue and assessing the binding of the ED-B fibronectin-specific molecule to the tumor cell or the tumor tissue, and
(b) stratifying said tumor cell or tumor tissue based on the assessment of step (a).

2. The method according to claim 1, wherein substantial binding of said ED-B fibronectin-specific molecule indicates sensitivity to anti-tumor treatment comprising administration of an EGF/EGFR antagonist.

3. The method of claim 1, wherein the tumor cell or tissue is selected from lung cancer, particularly NSCLC, or from prostate, breast, pancreas, colon, head-and-neck, gastric, ovarian, renal or cervix cancer.

4. The method of claim 1, wherein the tumor cell or tumor tissue is derived from or present in a human tumor patient.

5. The method of claim 4, which is carried out before the start of an anti-tumor treatment of said patient.

6. The method of claim 4, which is carried out during the anti-tumor treatment of said patient.

7. The method of claim 5, wherein said anti-tumor treatment comprises administration of an EGF/EGFR antagonist optionally in combination with the administration of further anti-tumor agents and/or radiation therapy.

8. The method of claim 7, wherein said EGF/EGFR antagonist is selected from small molecule EGFR inhibitors, particularly erlotinib or gefitinib.

9. The method of claim 7, wherein said EGF/EGFR antagonist is selected from anti EGFR antibodies, particularly cetuximab.

10. The method of claim 1, wherein said ED-B fibronectin-specific molecule is a peptide or an antibody.

11. The method of claim 10, wherein said ED-B fibronectin-specific molecule is an antibody having the CDR sequences from antibody L19.

12. The method of claim 11, wherein said ED-B fibronectin-specific molecule is the antibody AP39.

13. The method of claim 1, wherein said ED-B fibronectin-specific molecule is conjugated with a detectable labelling group, particularly with an imaging group.

14. The method of claim 13, wherein the labelling group is a radioactive labelling group.

15. The method of claim 13 wherein the labelling group is a SPECT labelling group, particularly ^{99m}Tc.

16. The method of claim 14, wherein the labelling group is a PET labelling group, particularly ¹⁸F.

17. The method of claim 1, which is carried out in vitro.

18. The method of claim 1, which is carried out in vivo.

19. A method of detecting epithelial mesenchymal transition (EMT) in a cell or tissue, particularly in a tumor cell or tumor tissue, comprising the steps of
(a) contacting an ED-B fibronectin-specific molecule with a cell or a tissue and assessing the binding of the ED-B fibronectin-specific molecule to the cell or tissue, and
(b) identifying said cell or tissue as epithelial-type cell or tissue or as mesenchymal-type cell or tissue based on the assessment of step (a).

20. The method of claim 1, wherein the assessment in step (a) is based on an imaging procedure.

21. An ED-B fibronectin-specific molecule comprising contacting with a method of stratifying a tumor cell or a tumor tissue as being sensitive or refractory against treatment an EGF/EGFR antagonist.

22. The method of claim 21, wherein the tumor cell or tumor tissue is selected from lung cancer, particularly NSCLC, or from prostate, breast, pancreas, colon, head-and-neck, gastric, ovarian, renal or cervix cancer.

23. The method of claim 21, wherein said anti-tumor treatment comprises administration of an EGF/EGFR antagonist optionally in combination with the administration of further anti-tumor agents and/or radiation therapy.

24. A method of detecting epithelial mesenchymal transition comprising use of an ED-B fibronectin binding molecule.

25. A composition or kit for treating a tumor comprising AP39 conjugated with a detectable labelling group and an EGF/EGFR antagonist.

26. The composition or kit of claim 25, wherein the detectable labelling group is a radioactive labelling group, particularly a SPECT labelling group or a PET labelling group.

27. A method of treating tumors comprising administering a composition according to claim 25.

28. N-[6-(4-[¹⁸F]fluorobenzylidene)aminooxyhexyl]maleimide AP39 ([¹⁸F]FBAM-AP39)

29. N-succinimidyl 4-[¹⁸F]fluorobenzoate AP39 ([¹⁸F]SFB-AP39)


SEQUENCE LISTING (28 sequences)

SEQ ID NO: 1 - protein, 238 aa, Artificial Sequence
  Description of Artificial Sequence Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 2 - protein, 4 aa, Artificial Sequence
  Description of Artificial Sequence Synthetic peptide
XXXC

SEQ ID NO: 3 - protein, 5 aa, Artificial Sequence
  Description of Artificial Sequence Synthetic peptide
XXXCX

SEQ ID NO: 4 - protein, 6 aa, Artificial Sequence
  Description of Artificial Sequence Synthetic peptide
HHHHHH

SEQ ID NO: 5 - protein, 17 aa, Artificial Sequence
  Description of Artificial Sequence Synthetic peptide
AAADDDSDDDYKDDDDK

SEQ ID NO: 6 - protein, 23 aa, Artificial Sequence
  Description of Artificial Sequence Synthetic peptide
AAADDDSDDDYKDDDDKHHHHHH

SEQ ID NO: 7 - protein, 121 aa, Artificial Sequence
  Description of Artificial Sequence Synthetic polypeptide
SGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 8 - protein, 5 aa, Artificial Sequence
  Description of Artificial Sequence Synthetic peptide
SFSMS

SEQ ID NO: 9 - protein, 17 aa, Artificial Sequence
  Description of Artificial Sequence Synthetic peptide
SISGSSGTTYYADSVKG

SEQ ID NO: 10 - protein, 7 aa, Artificial Sequence
  Description of Artificial Sequence Synthetic peptide
PFPYFDY

SEQ ID NO: 11 - protein, 12 aa, Artificial Sequence
  Description of Artificial Sequence Synthetic peptide
RASQSVSSSFLA

SEQ ID NO: 12 - protein, 6 aa, Artificial Sequence
  Description of Artificial Sequence Synthetic peptide
YASSRA

SEQ ID NO: 13 - protein, 10 aa, Artificial Sequence
  Description of Artificial Sequence Synthetic peptide
CQQTGRIPPT

SEQ ID NO: 14 - protein, 4 aa, Artificial Sequence
  Description of Artificial Sequence Synthetic peptide
GGGC

SEQ ID NO: 15 - protein, 4 aa, Artificial Sequence
  Description of Artificial Sequence Synthetic peptide
GCGC

SEQ ID NO: 16 - protein, 5 aa, Artificial Sequence
  Description of Artificial Sequence Synthetic peptide
GGGCA

SEQ ID NO: 17 - protein, 5 aa, Artificial Sequence
  Description of Artificial Sequence Synthetic peptide
GCGCA

SEQ ID NO: 18 - protein, 6 aa, Artificial Sequence
  Description of Artificial Sequence Synthetic peptide
HHHHHH

SEQ ID NO: 19 - protein, 22 aa, Artificial Sequence
  Description of Artificial Sequence Synthetic peptide
MKYLLPTAAAGLLLLAAQPAMA

SEQ ID NO: 20 - protein, 242 aa, Artificial Sequence
  Description of Artificial Sequence Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGC

SEQ ID NO: 21 - protein, 243 aa, Artificial Sequence
  Description of Artificial Sequence Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGGGCA

SEQ ID NO: 22 - protein, 242 aa, Artificial Sequence
  Description of Artificial Sequence Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGCGC

SEQ ID NO: 23 - protein, 243 aa, Artificial Sequence
  Description of Artificial Sequence Synthetic polypeptide
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKGCGCA

SEQ ID NO: 24 - protein, 283 aa, Artificial Sequence
  Description of Artificial Sequence Synthetic polypeptide
MKYLLPTAAAGLLLLAAQPAMAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKAAADDDSDDDYKDDDDKHHHHHH

SEQ ID NO: 25 - protein, 365 aa, Artificial Sequence
  Description of Artificial Sequence Synthetic polypeptide
MXDTPAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKSGGSGGPRAAPEVYAFATPEWPGSRDKRTLACLIQNFMPEDISVQWLHNEVQLPDARHSTTQPRKTKGSGFFVFSRLEVTRAEWEQKDEFICRAVHEAASPSQTVQRAVSVNPESSRRGGC

SEQ ID NO: 26 - protein, 244 aa, Artificial Sequence
  Description of Artificial Sequence Synthetic polypeptide
MXDTPAEVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSGDGSSGGSGGASTGEIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIK

SEQ ID NO: 27 - protein, 243 aa, Artificial Sequence; identical to SEQ ID NO: 21
  Description of Artificial Sequence Synthetic polypeptide

SEQ ID NO: 28 - protein, 5 aa, Artificial Sequence
  Description of Artificial Sequence Synthetic peptide
HHHHH